(19) United States
(12) Patent Application Publication
(10) Pub. No.: US 2013/0184772 A1
(43) Pub. Date: Jul. 18, 2013

(54) CENTRAL CONTROL UNIT OF IMPLANTS

(71) Applicant: Peter Osypka, Grenzach-Wyhlen (DE)

(72) Inventor: Peter Osypka, Grenzach-Wyhlen (DE)

(21) Appl. No.: 13/810,011

(22) PCT Filed: Jul. 8, 2011
(86) PCT No.: PCT/EP2011/003406

§ 371 Date: Mar. 29, 2013

(30) Foreign Application Priority Data
Jul. 13, 2010 (DE) 20 2010 010 220.0

(51) Int. Cl. A61N 1/39; A61N 1/37

(52) U.S. Cl. CPC A61N 1/3962; A61N 1/37
USPC 607/4

(57) ABSTRACT

The entire electronics of a control system and of implantable passive and active medical implants connected to or cooperating with the control system are integrated in a central control unit (ZSE) which, by way of detachable cables or by telemetry or radio contact, controls and monitors all of the implants that are present and, if appropriate, implants that are fitted subsequently in the patient (2). The central control unit is powered by an exchangeable battery. An implant can be switched on or switched off as and when necessary.

### BACKGROUND

The invention relates to a control system and arrangement of implantable active medical implants, particularly pacemakers and defibrillators (ICD), including their electronics, voltage sources and electrodes.

In addition, medically passive implants can also be provided.

Pacemakers and defibrillators are presently produced and implanted within a common titanium-encapsulated housing together with the associated electronics and battery. Until the end of the 1970s, implantable pacemakers were the only electronic permanent implant in the human body, and the defibrillators (ICD) appeared later. With the increasing reliability and the integration of electronic circuits, there are today a number of other electronically controlled implants, such as nerve stimulators and bladder stimulators, but also implants that release medicaments or that simply perform purely monitoring functions (ECG storage). Although the complexity of today's pacemakers has afforded considerable advantages for patients, it is only experienced cardiologists who are able to adopt and use the many possible parameters optimally for the particular patient.

Since they take over vital functions, they are subject to strict quality assurance measures and are equipped with particularly high-quality components. For this reason, the failure probability of the electronics is particularly low.

When it is to be exchanged for example, the pacemaker, of which the circuitry and battery are located in a common hermetically sealed titanium housing, is completely replaced. This means that about 95% of the value of the still fully functional system is lost. After replacement, all the parameters have to be adapted anew to the patient. Therefore, when a normal battery runs out, a pacemaker is exchanged only in a hospital where the suitable programming equipment for this specific pacemaker is available and where there is an experienced cardiologist who optimally “adjusts” this to the patient again. This is particularly inconvenient for pacemaker patients who travel or take vacations abroad, where the necessary programming equipment is often unavailable. In recent years, the complexity has increased still further since, in addition to the pacemakers, a defibrillator (ICD) is now integrated within the same housing. In patients with recurring ventricular flutter, the battery capacity is of very great importance. The number of defibrillator shocks is limited depending on the battery capacity. Battery replacement nowadays means that the entire unit, that is to say pacemaker and defibrillator, has to be taken out and replaced. Thereafter, the patient has to be “adjusted” again by an experienced cardiologist with the requisite programming equipment, that is to say the unit has to be programmed according to his medical requirements, which often takes a great deal of time.

### SUMMARY

The object is therefore to make available a control system and arrangement of implantable active medical implants, particularly pacemakers and defibrillators (ICD), including their electronics, voltage sources and electrodes.

This object is achieved by the means and features of the invention. Preferred and advantageous embodiments are set forth below.

In the control system and arrangement of implants as defined in the introduction, provision is made, according to the invention, that they are divided into modules or separate units in proximity to the target area and are monitored and activated or controlled from a central control unit (ZSE).

The electronics and control system at least of one circuitry module can be integrated and monitored in the central control unit, wherein at least the electronics and control system of a pacemaker (circuitry module) can be integrated and monitored in this central control unit.

However, it is also possible or additionally possible that at least the electronics and control system (circuitry module) of a pacemaker and defibrillator are integrated and monitored in the central control unit.

In a further embodiment, provision can be made that at least a receiver (header) for pacemaker electrodes and defibrillation electrodes is integrated in the central control unit. The modules can be controlled by telemetry or by cable connection.

Through the use of the invention and the embodiments, it is possible that the batteries of the central control unit and/or of the modules can be easily exchanged separately, such that the actual implants can remain in the patient even when the battery is being exchanged.

The central control unit can be supplied with electrical energy by a built-in antenna from a transmitter located outside the body of the patient, such that a battery could be omitted. However, it is expedient if the central control unit can be powered in emergencies by an additional implanted battery.

According to the invention, the central control unit contains, in addition to the control system, at least one medical implant.

In an expedient embodiment, provision can be made that the central control unit and the modules or some of the modules can be operated or powered centrally from a battery or an accumulator.

In an expedient and advantageous embodiment, provision can be made that the entire electronics and control system of the implants are integrated in the central control unit which, by way of detachable cables or by telemetry, controls and monitors all of the implants in the patient. The central control unit can have a dedicated and easily exchangeable battery, such that the central control unit can remain in its position during the battery exchange.

In an expedient embodiment, an implant can be switched on or switched off as and when necessary, and the individual implanted modules preferably have a dedicated battery or a dedicated accumulator and only the minimal electronics needed to ensure that they can perform the activities predetermined by the central control unit.

The entire electronics and control system of the implants are integrated (as circuitry modules of electronic circuits) in a central control unit (ZSE) which, by way of detachable cables, but preferably by telemetry, controls and monitors all of the implants in the patient. The central control unit has a dedicated and easily exchangeable battery. An implant can be switched on or switched off as and when necessary. The individual modules can have a dedicated battery or accumulator and contain only the minimal electronics needed to ensure that they can perform the necessary activities predetermined by the central control unit (ZSE).

Implantable Cardiac Defibrillator (ICD)

To exchange a battery for the defibrillator, the invention allows, for example, the following procedure.

The circuitry of the defibrillator is situated in the central control unit. The battery and the charging capacitor of the defibrillator are situated in a separate housing, which is implanted in the patient at an easily accessible location, e.g. in the abdominal space below the apex of the heart, but which is connected to the central control unit by a releasable cable. A short distance from there, the electrodes for the defibrillator can be placed on or in the heart. From the information of the pacemaker, the central control unit would first establish the diagnosis of ventricular flutter and send the information to the separate defibrillator for supplying the defibrillation shock, i.e. charging of the capacitor. After charging is complete, the defibrillator receives the order, from the central control unit, to output an energy impulse, i.e. the defibrillator is equipped, in addition to the battery, with a minimum of electronics and is therefore cost-effective and easy to replace.

What are the Advantages of the Technical Features?

The most important advantage of this invention is that no reprogramming of the patient implant has to be carried out during battery exchange. This exchange can be carried out anywhere in the world by any physician or surgeon, and no experience of the mode of function of the device is needed. During the battery exchange, the housing with the battery is thus removed separately from the body by releasing the connection cable to the central control unit and replaced by a housing fitted with a fresh battery, such that exchange of the actual medical implant and of the central control unit can be omitted. Reprogramming and adaptation to the patient are thus dispensed with. With an international agreement (standardization), the central control unit can be produced in large batch numbers and therefore cost-effectively. This arrangement would also be an ideal solution for patients who initially require a pacemaker and later also have to be provided with a defibrillator (ICD), since the new implant is easily switched on from the central control unit in which the electronics of the defibrillator are already integrated. The defibrillator (ICD) can expediently be implanted below the apex of the heart, wherein the electrodes are placed externally on both sides of the heart. In each of these cases, the individual programming of the patient is retained in the central control unit and readjustment on the patient is dispensed with. It would be possible to proceed in a similar way with other electronically controlled implants.

The following examples are cited here:
ECG storage
Neurostimulation (deep brain stimulation)
Stimulator against incontinence
Release of medication, e.g. chemotherapy (port system)
Pump devices for medicines
Blood pressure monitoring
Stomach stimulation
Blood analysis
Glucose measurements (diabetes)
Measurement of cardiac output
Special atrial stimulation
Biventricular stimulation (resynchronization)
General patient monitoring (telemedicine, Internet)
Battery monitoring and/or battery charging (accumulator mode)
Defibrillation module (ICD)
Vagal stimulation

These devices are controlled by microprocessors and integrated circuits (circuitry modules). It is very easy to imagine the difficulties that arise when a number of these devices have been implanted in one patient nowadays, since this increasingly poses problems, for example as a result of said devices influencing one another.

A further benefit of the invention is that it is not just the defibrillator module that can be placed separately, but also the battery of the central control unit (ZSE). Considerable advantages are likewise afforded through simple exchange of the battery alone. Whereas one nowadays attempts, in some cases with great expenditure of time, to find the lowest possible stimulus threshold in the heart in order to protect the battery capacity or, for the same purpose, modify the electrode heads of the pacemaker electrodes by expensive coatings (IrOx) and additions of medicaments (steroids), this would no longer be completely necessary with easy exchange of the battery. On the one hand, the main module could be left in the body of the patient (no reprogramming), and, on the other hand, the starting impulse of the pacemaker could be increased in amplitude such that an always reliable stimulation of the heart is ensured. This would eliminate all “EXIT problems”.

In today's implantation of a pacemaker, the physician seeks to find the lowest possible stimulus threshold both in the atrium and also in the ventricle, in order to place the electrode there and thus save battery energy. This often requires a lot of time and patience. This procedure can be greatly simplified and shortened through the simple exchange of the battery and through the use of batteries of high capacity.

Energy Supply to the Central Unit:

The energy supply to the central control unit can also be effected via a separate exchangeable battery. The implantation site of the battery should be chosen such that the latter can be easily explanted and exchanged. The connection between battery and central control unit is effected via an insulated biocompatible electric cable.

Emergency Situation:

For emergency situations, e.g. when abroad or “away from civilization”, a receiver integrated in the central unit converts an externally applied magnetic field into an operating voltage and thus temporarily powers the central unit.

Disturbances from Outside:

If the implants are affected by very strong external electromagnetic disturbances and can thus fail, it is possible for the patient to wear, for protection, an item of clothing (e.g. shirt) of which the fibers are electrically conductive and thus constitute a “Faraday cage”.

Further Advantages:

Great advantages are afforded in particular when module dimensions, electronics and control systems are standardized. If a battery runs out and the implant therefore has to be exchanged, it is no longer necessary to exchange the expensive electronics too. The entire electronics (electronic circuitry modules) and control system are accommodated in a hermetically tight housing and are intended to remain functional for the whole remaining lifespan of the patient. The price can be considerably lowered through mass production.

A simple surgical intervention permits simple exchange of the battery, for example. Subsequent programming is no longer necessary by specialists and can therefore be carried out worldwide. This avoids possible limitation of the patient's mobility and travel opportunities. Moreover, for long periods of planned absence, every patient can carry around a sterile replacement battery in an implantable housing (lithium batteries have a very low self-discharge and can be stored for a long time), which battery can then easily be exchanged by a surgeon as and when necessary.

It would be possible to implant a replacement battery too (e.g. with low power and only for emergency requirements), which can then be switched on, as and when necessary, by a programmable switch. An antenna (coil) to be placed on the patient's body can also deliver energy to the central control unit via a “transmitter”. All the data and measurement values of the individual modules can be transmitted by telemetry to the outside and via the normal communication paths (e.g. telephone). The remote monitoring of the patient by telemetry and Internet can also be achieved in this way and transferred via the normal communication paths (e.g. telephone).

Summary of the Advantages:
Easy and inexpensive exchange of the battery, no reprogramming.
Possibilities of battery charging (for special uses)
Outpatient operation, also possible in general practices (no specialists needed)
Less need for a lower stimulus threshold, since battery energy part easily exchangeable
Easy and inexpensive exchange of modules when defects occur or in the event of improvements of the technology.
Through separate delivery and storage of central control unit and battery, the overall lifetime of the system can be extended since, during storage, there is no “quiescent current” flow, as is the case in today's implants.
The development of planned new 4-pin IS4 plugs is no longer necessary. This makes storage in hospitals easier, since a large number of adapters, with all of their possible faults and difficulties, would be dispensed with.

### BRIEF DESCRIPTION OF THE DRAWINGS

Further details, features and advantages of the invention will become clear from the following part of the description, in which the invention is explained in more detail with reference to the drawings and illustrative embodiments. These show in schematic representation:

FIG. 1 shows a block diagram of the central control unit (ZSE) with an integrated pacemaker module, e.g. a four-chamber pacemaker.

FIG. 2 shows schematically the central control unit (ZSE), in which the battery required for it is placed separately in an easily accessible operating area.

FIG. 3 shows schematically the position of the defibrillator module with the electrodes fixed directly on both sides of the heart or in proximity thereto.

FIG. 4 shows schematically the central control unit with a number of modules in the human body.

FIG. 5 shows schematically an overview of implantable modules which can each be switched on and off and monitored and controlled from a central control unit.

### DETAILED DESCRIPTION OF THE PREFERRED EMBODIMENTS

A possible configuration of the central control unit is to integrate the entire electronics (modular electronic circuitry) and control system for a universal programmable pacemaker that covers all the necessary and possible stimulation forms and parameters. It is possible to activate each chamber of the heart separately, or also together, by simple switching on and off.

The energy can be supplied by an easily exchangeable battery in a common housing, or in two housings that are spatially separate in the position of use.

The receiving part of the electrodes (header) can likewise be accommodated in the common housing of the central control unit. This is particularly advantageous for easy transvenous implantation of the stimulation electrodes into the atrium and into the ventricle.

FIG. 2 shows how the battery can be placed in an area of the body that is as easy to access as possible, such that a necessary battery exchange can be easily carried out. The circuitry in the central control unit is configured such that the battery exchange does not make reprogramming (of the circuitry modules) necessary. It is thus ensured that a battery exchange can also be easily carried out by untrained pacemaker specialists. In any case, the expensive part, namely the central control unit (with all the circuitry modules), remains implanted in the patient's body.

FIG. 3 shows schematically the position of the defibrillator unit (which contains only the minimal electronics, as the actual defibrillator module sits in the central control unit) with the electrodes fixed directly on both sides of the heart or in proximity thereto. Here too, an easily accessible location in the human body will be sought such that, on the one hand, a battery exchange is easy to carry out and, on the other hand, the positioning and fixing of the defibrillation electrodes can be placed optimally, which is possible, for example, on both sides of the chambers of the heart. It is not absolutely essential that the electrodes are fixed directly on the epicardium of the heart, and instead it is sufficient if the electrodes are placed in proximity thereto such that, during the defibrillation, the current, if possible, flows through the whole heart.

FIG. 4 shows schematically the central control unit with a number of (circuitry) modules in the human body. The figure is intended to show that the communication between the central unit (ZSE) and the modules can be effected both by telemetry and by cable connection.

FIG. 5 shows schematically an overview of implantable modules that can each be switched on and off and monitored and controlled from a central control unit.

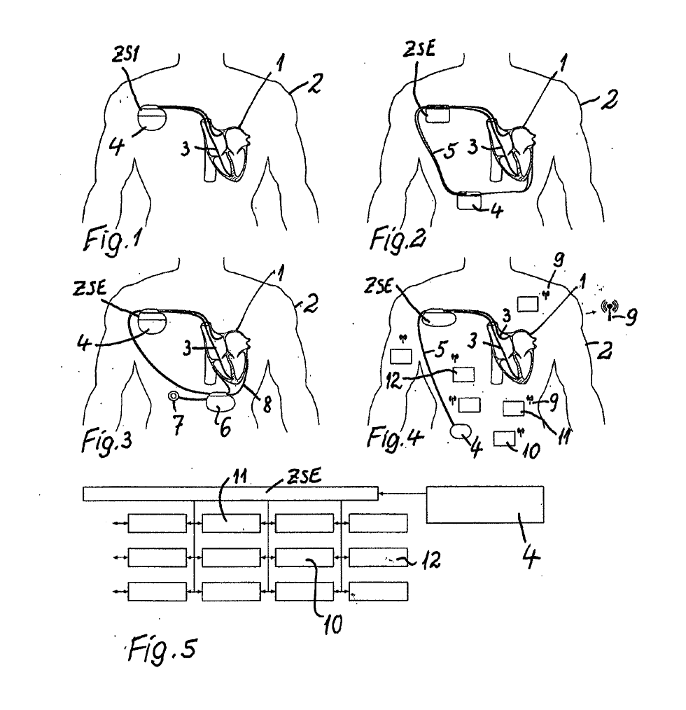

In FIGS. 1 to 4, the heart 1 of a patient 2 is shown schematically in longitudinal section, wherein a pacemaker electrode 3 in all four illustrative embodiments is inserted into the right ventricle of the heart 1.

In FIG. 1, this pacemaker electrode 3 comes from a central control unit (ZSE) arranged in a common housing in accordance with the above description. This has a separate housing part 4 for an exchangeable battery, which can therefore be removed and exchanged after it has run out, without likewise having to exchange the central control unit with an integrated pacemaker and/or defibrillator.

The arrangement according to FIG. 2 differs from the arrangement according to FIG. 1 in that the housing part 4 for the battery is arranged spatially at a distance from the central control unit (ZSE), from which pacemaker electrodes 3 again lead to the heart 1, and which is connected by a line 5 to the battery in the battery housing 4.

As regards the central control unit, FIG. 3 again shows an arrangement as in FIG. 1, but with the additional provision of a defibrillator 6, which has an antenna 7 and acts with its electrodes 8 on the outer face of the heart 1.

FIG. 4 shows an arrangement according to the invention with a central control unit (ZSE), from which pacemaker electrodes 3 run into the heart 1 of the patient 2. The housing part 4 for the battery is again arranged at a distance from the central control unit and is connected to the latter by a line 5.

By radio, various implants are connected to the central control unit (ZSE), and to the control modules contained therein, by radio contact, as is indicated by corresponding symbols 9. An implant 10 can serve for ECG monitoring, for example. A further implant 11 can be provided as defibrillator. An implant 12 can serve for dispensing medicaments, and so forth.

FIG. 5 shows, again in a schematic representation, the division of the central control unit (ZSE) according to the invention and its connection to the corresponding implant modules, wherein the housing part 4 for the battery for powering the central control unit is also indicated and illustrated schematically alongside this central control unit. The connection lines leading from the central control unit to the individual implants illustrate the modular configuration of the entire control system and arrangement of implantable implants, the central control unit also containing a pacemaker, which acts on the pacemaker electrode 3. Examples of implants, from “ECG storage” to “Vagal stimulation”, are mentioned earlier in the description.

The entire electronics of a control system, and of implantable passive and active medical implants connected to or cooperating with the control system, are integrated in a central control unit (ZSE) which, by way of detachable cables or by telemetry or radio contact, controls and monitors all of the implants that are present and, if appropriate, implants that are fitted subsequently in the patient 2. The central control unit is powered by an exchangeable battery. An implant can be switched on or switched off as and when necessary.

1. A control system for implantable active medical implants, including their electronics, voltage sources and electrodes, comprising implants that are divided into modules and are monitored and controlled from a central control unit (ZSE), the central control unit (ZSE) has a separate housing part in which a battery is located.

2. The control system as claimed in claim 1, wherein the electronics and control system at least of one of the modules are integrated and monitored in the central control unit.

3. The control system as claimed in claim 1, wherein at least the electronics and control system of a pacemaker are integrated and monitored in the central control unit (ZSE).

4. The control system as claimed in claim 1, wherein at least the electronics and control system of a pacemaker and defibrillator are integrated and monitored in the central control unit (ZSE).

5. The control system as claimed in claim 1, wherein at least a receiver for pacemaker electrodes and defibrillation electrodes is integrated in the central control unit (ZSE).

6. The control system as claimed in claim 1, wherein the modules are controlled by telemetry.

7. The control system as claimed in claim 1, wherein the modules are controlled by cable connection.

8. The control system as claimed in claim 1, wherein the battery of the central control unit (ZSE) is separately exchangeable.

9. The control system as claimed in claim 1, wherein the central control unit (ZSE) includes a built-in antenna, and can be supplied with electrical energy by the built-in antenna from a transmitter located outside the body.

10. The control system as claimed in claim 1, further comprising an additional implanted battery, and the central control unit (ZSE) can be powered in emergencies by the additional implanted battery.

11. (canceled)

12. The control system as claimed in claim 1, wherein the central control unit (ZSE) and the modules or some of the modules can be operated centrally from a battery or an accumulator.

13. The control system as claimed in claim 1, wherein the entire electronics and control system of the implants are integrated in the central control unit (ZSE) which, and the separate housing part that contains the battery is connected to the central control unit by detachable cables.

14. (canceled)

15. The control system as claimed in claim 1, wherein one of the implants can be switched on or switched off as and when necessary, and the individual implanted modules have a dedicated battery or a dedicated accumulator and only minimal electronics needed to ensure that the individual modules can perform the activities predetermined by the central control unit (ZSE).

16. The control system of claim 1, wherein the batteries of the modules are separately exchangeable.
